(19) United States
(12) Patent Application Publication
(10) Pub. No.: US 2013/0150640 A1
(43) Pub. Date: Jun. 13, 2013

(54) PRODUCTION OF XYLENES BY METHYLATION OF AROMATIC COMPOUNDS

(71) Applicant: GTC Technology US, LLC, Houston, TX (US)

(72) Inventors: Zhongyi Ding, Katy, TX (US); Amy Estes, Sugar Land, TX (US); Lucia Cretoiu, Sugar Land, TX (US); Mircea Cretoiu, Sugar Land, TX (US); Michael McCaulley, Sugar Land, TX (US)

(73) Assignee: GTC Technology US, LLC, Houston, TX (US)

(21) Appl. No.: 13/708,249

(22) Filed: Dec. 7, 2012

Related U.S. Application Data
(60) Provisional application No. 61/568,313, filed on Dec. 8, 2011.

(51) Int. Cl. C07C 2/86

(52) U.S. Cl. CPC C07C 2/864
USPC 585/300; 585/316; 585/450

(57) ABSTRACT

The inventive method is directed to the production of xylenes by methylation of aromatic compounds with methanol. The process uses fixed bed reactors, operates at lower pressure, and without the need for hydrogen or other gas recycle.

### CROSS-REFERENCES TO RELATED APPLICATIONS

This Application claims the benefit under 35 U.S.C. § 119(e) of U.S. Provisional Patent Application Ser. No. 61/568,313 filed Dec. 8, 2011, which is incorporated herein by reference in its entirety as if fully set forth herein.

### FIELD OF THE INVENTION

The claimed invention relates to a process for producing xylenes through the methylation of aromatic compounds using methanol.

### BACKGROUND OF THE INVENTION

Paraxylene is a valuable chemical intermediate used in the production of terephthalic acid, which in turn is used in the production of polymers such as polytrimethyleneterephthalate (PTT), polybutyleneterephthalate (PBT) and polyethyleneterephthalate (PET). Given the large market for PET plastics and fibers, in addition to other end products produced from paraxylene, there is a substantial demand for paraxylene in high purity

Catalytic reforming is a process by which aromatic compounds are produced in the petrochemical industry by the conversion of a naphtha hydrocarbon feed. In addition to mixed xylenes, the reforming process also produces benzene and toluene. To maximize paraxylene production by utilizing the aromatic compounds from the reforming process, the shortage of available methyl groups must be addressed. Aromatic methylation is an effective means to increase methyl groups on the aromatic ring and thereby maximize the production of mixed xylenes and paraxylene.

Prior art processes that are used to convert aromatic compounds utilize conditions that require high concentrations of hydrogen in the feedstock and also require the recycling of hydrogen and other gases during the conversion process, which renders these processes expensive and cost-inefficient. Thus, there is a need for an energy-efficient process that converts aromatic compounds to xylene compounds via methylation that does not require the recycling of hydrogen or other gases.

### SUMMARY OF THE INVENTION

An embodiment of the invention is directed to a process for producing xylenes by methylating aromatic compounds using methanol. The process uses fixed bed reactors, operates at lower pressure, and without the need for hydrogen or other gas recycle. Significant savings in energy for gas recycle make the aromatics methylation process more efficient than other processes known in the prior art.

Another embodiment of the invention is directed to a method for producing xylenes comprising the steps of: loading a zeolite catalyst into a fixed bed reactor system; feeding a feedstock to the fixed bed reactors, wherein the feedstock comprises at least one aromatic compound, methanol and water; reacting the feedstock in the presence of the zeolite catalyst to form an effluent, wherein the effluent comprises water, aromatic hydrocarbons, and light hydrocarbons; cooling the effluent; feeding the cooled effluent into a separator; separating a vapor phase stream, an aqueous stream, and hydrocarbon stream in the separator; distilling the hydrocarbon stream in a distillation section to form a product fraction and a fraction containing unreacted aromatic compounds; recycling a portion of the fraction containing unreacted aromatic compounds in the aqueous stream to the fixed bed reactor system; and diverting the vapor phase stream away from the fixed bed reactor system.

### BRIEF DESCRIPTION OF THE DRAWINGS

FIG. 1 is a process scheme in accordance with an embodiment of the invention;

FIG. 2 shows an arrangement of reactors in series for a process scheme in accordance with an embodiment of the invention; and

FIG. 3 shows an arrangement of reactors in parallel for a process scheme in accordance with an embodiment of the invention.

### DETAILED DESCRIPTION OF EXEMPLARY EMBODIMENTS

An embodiment of the invention is directed to a method for producing xylenes comprising the steps of: loading a zeolite catalyst into to a fixed bed reactor system; feeding a feedstock to the fixed bed reactors, wherein the feedstock comprises at least one aromatic compound, methanol and water; reacting the feedstock in the presence of the zeolite catalyst to form an effluent, wherein the effluent comprises water, aromatic hydrocarbons, and light hydrocarbons; cooling the effluent; separating a vapor phase stream from an aqueous stream and a hydrocarbon stream in a separator; distilling the hydrocarbon stream to form a product fraction and a fraction containing unreacted aromatic compounds; recycling a portion of the fraction containing unreacted aromatic compounds and methanol in the aqueous stream to the fixed bed reactors; and diverting the vapor phase stream away from the fixed bed reactor system. In this embodiment, the vapor phase stream or off gas is not recycled back into the feedstock or reactor system.

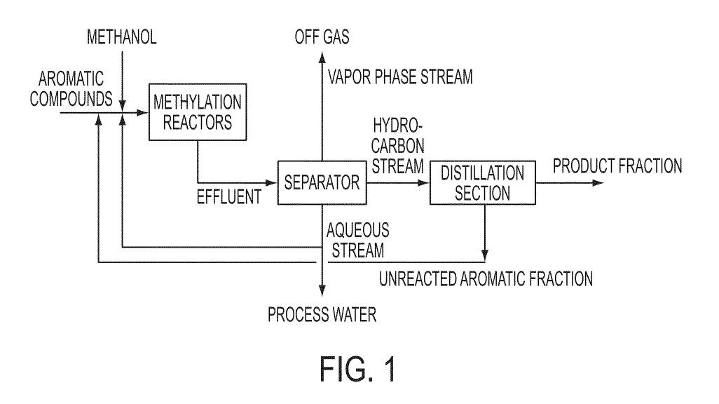

As set forth in FIG. 1, a mixture of methanol and aromatic compounds are fed into methylation reactors containing a zeolite catalyst. The effluent that is formed in the methylation reactors is fed into a separator where a vapor phase stream, an aqueous phase stream and a hydrocarbon phase stream are separated. The hydrocarbon phase stream is fed into a distillation section to form a product fraction comprising xylenes. The unreacted aromatic fraction is fed back into reactor system. In certain embodiments of the invention, an unreacted methanol fraction is removed from the distillation section and is concentrated and fed back into reactor system, along with the water (aqueous stream) in the reactor effluent.

In certain embodiments of the invention, the fixed bed reactor system comprises a single or a plurality of fixed reactors, where the reactors may be arranged in series or parallel.

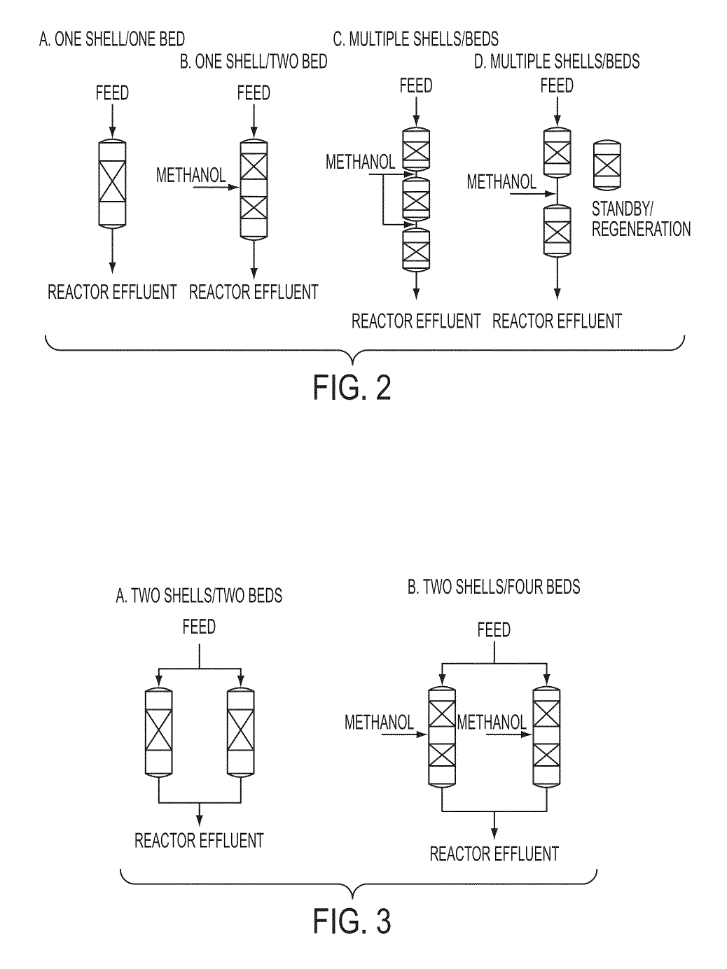

As set forth in FIGS. 2 and 3, the reactor system used in the inventive process can be designed in any number of ways to accommodate specific process conditions. In certain embodiments, the reactor system comprises a single shell with a single bed (FIG. 2A). In other embodiments, the reactor system comprises a single shell having a plurality of beds (FIG. 2B) in which the aromatic compounds and the methanol are fed into the reactor system through different input points. FIGS. 2C and 2D show multiple shell reactor systems connected in series including the use of a standby shell. FIG. 3 shows a multi-shell, multi-bed format where the reactors are connected in parallel.

In an embodiment of the invention, the inventive method is carried out at a temperature of 420-600° C. and pressure of 10-100 psig. In other embodiments of the invention, method is carried out at a temperature of 480-550° C. and pressure of 20-50 psig. In some embodiments of the invention, the WHSV of the claimed method is in the range of 2-12 hr⁻¹. In a preferred embodiment the WHSV of the method is in the range of 4-8 hr⁻¹.

In an embodiment of the invention, the aromatic compound that is used in the feedstock is selected from the group consisting of benzene, toluene or a mixture benzene and toluene. In certain embodiments of the invention, the feedstock also comprises hydrogen at a concentration of less than 10 mole %. In certain embodiments of the invention, the aromatic compound(s) in the feedstock are presentation at a concentration of 40 wt % to 90 wt %.

In certain embodiments of the invention, the zeolite catalyst is selected from the group consisting of zeolites X, Y and beta, mordenite, silico-alumino-phosphate, H-ZSMS, ZSM-5, ZSM-11, TS-1, Fe-silicalite, TNU-9 and HIM-5.

In embodiments of the invention, the zeolite catalyst that is used is either ZSM-5 that is modified by at least one element selected from sodium, magnesium, barium, boron, phosphorus and platinum; ZSM-5 that is modified by silylation with organic silicon; ZSM-5 bound with silica, alumina, magnesium silica or clay; or ZSM-5 that is combined with a zeolite binder.

In certain embodiments of the invention, the zeolite catalyst is ZSM-5 having a silica to alumina ratio in the range of 150-450, and more preferably in the range of 200-300.

In some embodiments of the invention, the zeolite catalyst is regenerated upon completion of the end of the run of the xylene production process. In some embodiments, the zeolite catalyst is regenerated in situ within the fixed bed reactor system by oxidation. In certain embodiments of the invention, the oxidation process is carried out using a stream of diluted oxygen.

In an embodiment of the invention, the feedstock comprises at least one aromatic compound and methanol in a ratio ranging from 1:1 to 10:1. In some embodiments, the ratio range from 2:1 to 8:1, and 3:1 to 6:1.

In an embodiment of the invention, the product fraction comprises a mixture of xylenes that are present at 70 wt % to 95 wt % of the product fraction, and more preferably at 80 wt % to 95 wt % of the product fraction. The paraxylene selectivity in the mixed xylenes ranges from 25 wt % to 95 wt % and more preferably from 40 wt % to 87 wt %.

In an embodiment of the invention, the conversion of the aromatic compounds in the feedstock obtained using the claimed method ranges from 8 wt % to 40 wt % and more preferably from 15 wt % to 35 wt %. In certain embodiments of the invention, the conversion of the aromatic compounds in the feedstock ranges from 20 wt % to 30 wt %.

### WORKING EXAMPLES

### Example 1

A case study was conducted to determine the energy savings realized in the absence of having to recycle hydrogen gas. A 400 KTA (kilo tonnes per year) toluene feed was fed into a process scheme in accordance with an embodiment of the invention. Three reactors were placed in series. The total conversion of toluene to paraxylene obtained was 30%. The pressure at the first reactor was 80 psig and the final pressure before recycle compressor was measured at 20 psig. The ratio of H₂/Toluene=2 mol/mol at the first reactor. If hydrogen recycle was included in the process scheme, the toluene feed rate to the reactor would be 1333 KTA (equivalent to 14.5 KT mol/year or 14.5×10⁶ kg-mol/year). Furthermore, the hydrogen that would be required to be recycled would be 29.0×10⁶ kg-mol/year. The energy requirements for recycling the hydrogen would be 32×10⁶ kW/year or $3.2 million dollars with the electricity at $0.1/kW, in addition to having to consider how to utilize the hydrogen. Since the claimed process does not incorporate a hydrogen recycle step, a significant savings in energy costs is realized.

### Example 2

Three time silylated ZSM-5 on silica binder (SAR 250-300) was prepared as a catalyst. 15 grams of catalyst was loaded in a fixed bed reactor. Toluene methylation was tested under the following conditions: Toluene: Methanol mole ratio =8:1, H₂=0, H₂O/hydrocarbons (HC)=1, WHSV=4 h⁻¹; Pressure=30 psig; Temperature=480° C. Toluene conversion under these conditions was 9.1 mol %, paraxylene (PX) selectivity was 71.2 mol %, and methanol utilization was 66 mol %.

Example 3

Three time silylated ZSM-5 on silica binder (SAR 250-300) was prepared as a catalyst. 10 grams of catalyst was loaded in a fixed bed reactor. Toluene methylation was tested under the following conditions: Toluene: Methanol mole ratio=4:1, H₂=0, H₂O/HC=1, WHSV=4 h⁻¹; Pressure=0 psig; Temperature =480° C. Toluene conversion was 14.1 mol %, PX selectivity was 73.6 mol %, and methanol utilization was 58.8 mol %.

### Example 4

One time silylated ZSM-5 on silica binder (SAR 250-300) was prepared as a catalyst. 10 grams of catalyst was loaded in a fixed bed reactor. Toluene methylation was tested under the following conditions: Toluene: Methanol mole ratio=4:1, H₂=0, H₂O/HC=1, WHSV=4 h⁻¹; Pressure=30 psig; Temperature =480° C. Toluene conversion was 17.0 mol %, PX selectivity was 40.7 mol %, and methanol utilization was 57.4 mol %.

Example 5

Three time silylated ZSM-5 on silica binder (SAR 150-200) was prepared as a catalyst. 10 grams of catalyst was loaded in a fixed bed reactor. Toluene methylation was tested under the following conditions: Toluene: Methanol mole ratio=4:1, H₂=0, H2O/HC=1, WHSV=4 h⁻¹; Pressure=0 psig; Temperature =480° C. Toluene conversion was 12.5 mol %, PX selectivity was 85.2 mol %, and methanol utilization was 43.8 mol %.

Example 6

ZSM-5 (SAR250-300) on alumina binder was prepared as a catalyst. 2 grams of catalyst was loaded in a fixed bed reactor. Toluene methylation was tested under the following conditions: Toluene: Methanol mole ratio=4:1, H₂=0, H₂O/HC=1, WHSV=4 h⁻¹; Pressure=0 psig; Temperature=480° C. Toluene conversion was 25.2 mol %, PX selectivity was 27.6 mol %, and methanol utilization was 70 mol %.

Example 7

Barium ion exchanged ZSM-5 (SAR 250-300) was prepared as a catalyst. 2 grams of this catalyst powder was loaded in a fixed bed reactor. Toluene methylation was tested under the following conditions: Toluene: Methanol mole ratio=4:1, H₂=0, H2O/HC=1, WHSV=10 h⁻¹; Pressure=0 psig; Temperature =500° C. Toluene conversion was 18 mol %, PX selectivity was 50 mol %, and methanol utilization was 65 mol %.

Example 8

Phosphorus impregnated ZSM-5 was silylated with TOES and supported on silica to form a catalyst. 4 grams of the catalyst was loaded in a fixed bed reactor. Toluene methylation with dimethyl ether (DME) was tested under the following conditions: Toluene: DME mole ratio=4:1, H₂=0, H₂O/HC=1, WHSV=10 h⁻¹; Pressure=0 psig; Temp=480° C. Toluene conversion was 13 mol %, PX selectivity was greater than 90 mol %, and methanol utilization was 48 mol %.

Example 9

One time silylated ZSM-5 on silica binder (SAR 250-300) was prepared as a catalyst. 10 grams of catalyst was loaded in a fixed bed reactor. Toluene methylation was tested under the following conditions: Toluene: Methanol mole ratio=4:1, H₂=0, H2O/HC=1, WHSV=4 h⁻¹; Pressure=30 psig; Temperature=480° C. and ethylene flow to the reactor was 10 ml/min. Toluene conversion was17.5 mol %, PX selectivity was 40 mol %, and methanol utilization was 55 mol %.

Example 10

ZSM-5 on silica binder (SAR 250-300) was prepared as a catalyst. 2 grams of catalyst was loaded in a fixed bed reactor. Toluene methylation was tested under the following conditions: Toluene: Methanol mole ratio=4:1, H₂=0, H₂O/HC=1, WHSV=4 h⁻¹; Pressure=0 psig; Temperature=480° C. Toluene conversion was 21 mol %, PX selectivity was 44 mol %, and methanol utilization was 72 mol %.

Example 11

Three time silylated ZSM-5 on silica binder (SAR 250-300) was prepared as a catalyst. 15 grams of catalyst was loaded in a fixed bed reactor. Benzene methylation was tested under the following conditions: Benzene: Methanol mole ratio=8:1, H₂=0, H2O/HC=1, WHSV=4 h⁻¹; Pressure=30 psig; Temp =480° C. Benzene conversion was 9 mol %, toluene to mixed xylenes ratio was about 10/1, and methanol utilization was about 74 mol %.

Although the present invention has been described in connection with some embodiments, it is not intended to be limited to the specific form set forth herein. Additionally, although a feature may appear to be described in connection with particular embodiments, one skilled in the art would recognize that various features of the described embodiments may be combined in accordance with the invention. In the claims, the term comprising does not exclude the presence of other elements or steps.

What is claimed is:

1. A method for producing xylenes comprising the steps of:
a. loading a zeolite catalyst into a fixed bed reactor system;
b. feeding a feedstock to the fixed bed reactors, wherein the feedstock comprises at least one aromatic compound, methanol and water;
c. reacting the feedstock in the presence of the zeolite catalyst to form an effluent, wherein the effluent comprises water, aromatic hydrocarbons, and light hydrocarbons;
d. cooling the effluent;
e. feeding the cooled effluent into a separator;
f. separating a vapor phase stream, an aqueous stream, and hydrocarbon stream in the separator;
g. distilling the hydrocarbon stream in a distillation section to form a product fraction and a fraction containing unreacted aromatic compounds;
h. recycling a portion of the fraction containing unreacted aromatic compounds to the fixed bed reactor system; and
i. diverting the vapor phase stream away from the fixed bed reactor system.

2. The method of claim 1, wherein the fixed bed reactor system comprises a single or a plurality of fixed reactors.

3. The method of claim 2, wherein the plurality of reactors is arranged in series.

4. The method of claim 2, wherein the plurality of reactors is arranged in parallel.

5. The method of claim 1, wherein the fixed bed reactor system is operated at a temperature of 420-600° C. and pressure of 10-100 psig.

6. The method of claim 1, wherein the fixed bed reactor system is operated at a temperature of 480-550° C. and pressure of 20-50 psig.

7. The method of claim 1, wherein the WHSV is in the range of 2-12 hr⁻¹.

8. The method of claim 1, wherein the WHSV is in the range of 4-8 hr⁻¹.

9. The method of claim 1, wherein the at least one aromatic compound is selected from the group consisting of benzene, toluene and a mixture benzene and toluene.

10. The method of claim 1, wherein the feedstock further comprises hydrogen.

11. The method of claim 1, wherein the zeolite catalyst is selected from the group consisting of zeolite X, zeolite Y, beta, mordenite, SAPO, H-ZSM5, ZSM-5, ZSM-11, TS-1, Fe-silicalite, zeolite TNU-9 and zeolite HIM-5.

12. The method of claim 1, wherein the zeolite catalyst is ZSM-5 that is modified by at least one element selected from sodium, magnesium, barium, boron, phosphorus and platinum.

13. The method of claim 1, wherein the zeolite catalyst is ZSM-5 that is modified by silylation with organic silicon.

14. The method of claim 1, wherein the zeolite catalyst is ZSM-5 combined with silica, alumina, magnesium silica or clay.

15. The method of claim 1, wherein the zeolite catalyst is ZSM-5 that is combined with a zeolite binder.

16. The method of claim 1, wherein the zeolite catalyst is ZSM-5 having a silica to alumina ratio in the range of 150-450.

17. The method of claim 1, wherein the zeolite catalyst is ZSM-5 having a silica to alumina ratio in the range of 200-300.

18. The method claim 1 further comprising the step of regenerating the zeolite catalyst.

19. The method of claim 18, wherein the zeolite catalyst is regenerated in situ.

20. The method of claim 19, wherein the zeolite catalyst is regenerated by oxidation.

21. The method of claim 1, further comprising the step of recycling a portion of the unreacted methanol from the distillation section to the fixed bed reactor system.

22. The method of claim 1 wherein the feedstock comprises aromatic compounds and methanol in a ratio ranging from 1:1 to 10:1.

23. The method of claim 1 wherein the feedstock comprises aromatic compounds and methanol in a ratio ranging from 2:1 to 8:1.

24. The method of claim 1 wherein the feedstock comprises aromatic compounds and methanol in a ratio ranging from 3:1 to 6:1.

25. The method of claim 1, wherein the product fraction comprises a mixture of xylenes.

26. The method of claim 25 wherein the mixture of xylenes is present at 70% to 95% of the product fraction.

27. The method of claim 25 wherein the mixture of xylenes is present at 80% to 90% of the product fraction.

28. The method of claim 25 wherein, the paraxylene selectivity in mixed xylenes is in the range of 25% to 95%.

29. The method of claim 25 wherein, the paraxylene selectivity in mixed xylenes is in the range of 40% to 87%.

30. The method of claim 1, wherein the conversion of the aromatic compounds in the feedstock ranges from 8 wt % to 40 wt %.

31. The method of claim 1, wherein the conversion of the aromatic compounds in the feedstock ranges from 15 wt % to 35 wt %.

32. The method of claim 1, wherein the conversion of the aromatic compounds in the feedstock ranges from 20 wt % to 30 wt %.
